(19) United States
(12) Patent Application Publication
(10) Pub. No.: US 2025/0134414 A1
(43) Pub. Date: May 1, 2025

(54) METHODS AND SYSTEMS FOR DETECTING EVENTS USING ACTIGRAPHY DATA

(71) Applicant: Eli Lilly and Company, Indianapolis, IN (US)

(72) Inventors: Yu-Min CHUNG, Fishers, IN (US); Ju JI, Carmel, IN (US); Amir NIKOOIE, Carmel, IN (US); Bo ZHANG, Bloomington, IN (US)

(21) Appl. No.: 18/837,738

(22) PCT Filed: Jan. 30, 2023
(86) PCT No.: PCT/US23/11831

§ 371 Date: Aug. 12, 2024

Related U.S. Application Data
(60) Provisional application No. 63/310,835, filed on Feb. 16, 2022.
(60) Provisional application No. 63/354,061, filed on Jun. 21, 2022.

(51) Int. Cl. A61B 5/11; A61B 5/00; G16H 10/60; G16H 20/60; G16H 80/00

(52) U.S. Cl. CPC A61B 5/1123; A61B 5/0022; A61B 5/4866; A61B 5/681; G16H 10/60; G16H 20/60; G16H 80/00; A61B 2562/0219

(57) ABSTRACT

A computerized method is provided for detecting whether a subject is experiencing an event (e.g., an eating event or a scratching event). The method may comprise receiving time-series data derived from data recorded by an actigraphy device worn on a wrist of the subject and computing a set of birth and death coordinates for each topological feature of a plurality of topological features in the received time-series data. The method may further comprise calculating a digital feature based on the computed set of birth and death coordinates and determining whether the subject is experiencing the event based on the calculated digital feature. In some embodiments, methods are also provided for using Bayesian methods for selecting digital features to use in detecting whether the subject is experiencing the event.

### FIELD OF THE DISCLOSURE

The present disclosure relates to methods and systems for detecting events using actigraphy data. More particularly, the present disclosure relates to using accelerometer and/or gyroscope data recorded by an actigraphy device worn on a wrist of a human subject to detect whether the subject is or has performed certain actions or events.

### BACKGROUND OF THE DISCLOSURE

A subject's movements may be detected and/or measured by wearable actigraphy devices. This movement data may be analyzed to determine whether the subject is performing, or has performed, certain actions or events.

For persons with diabetes, it is sometimes important to inject a bolus of insulin shortly before or after ingesting food in order to prevent a harmful rise in glucose levels, i.e., a hyperglycemic episode. However, persons with diabetes frequently miss their bolus insulin doses because they simply forget to inject themselves before or after eating food. This can make it more difficult for such persons to maintain their glucose levels in a healthy range.

For persons looking to lose weight, it may be desirable to maintain an accurate log of food ingested. Such a log can help such persons as well as their healthcare providers identify opportunities for decreasing consumption of food, and guide decisions regarding what and when to eat.

Therefore, a need exists for methods and devices to automatically detect, without requiring user input, when a person is eating.

Persons who are suffering from certain skin conditions may exhibit scratching behavior. Such persons may scratch themselves during the day, while awake, or at night while asleep. Maintaining a log of the incidence and/or severity of scratching behavior may be useful for detecting such skin conditions, assessing the severity of such skin conditions, or evaluating the progression of such skin conditions. Therefore, a need also exists for methods and devices to automatically detect, without requiring user input, when a person is scratching.

### SUMMARY

According to an exemplary embodiment of the present disclosure, a computerized method for detecting whether a subject is experiencing an event using one or more processors is provided, the method comprising: receiving time-series data derived from data recorded by an actigraphy device worn on a wrist of the subject; computing a set of birth and death coordinates for each topological feature of a plurality of topological features in the received time-series data; calculating a digital feature based on the computed set of birth and death coordinates for each topological feature in the plurality of topological features; and determining whether the time-series data indicates the subject is experiencing the event based on the calculated digital feature.

According to another embodiment of the present disclosure, a computerized method for training a machine-learning model to detect whether a subject is experiencing an event using one or more processors is provided, the method comprising: receiving, training data comprising: a plurality of sets of time-series data derived from data recorded by an actigraphy device worn on a wrist of a subject, and a ground truth label associated with each respective set of time-series data indicating whether the respective set of data is derived from data recorded by the actigraphy device while the subject is experiencing the event; calculating a plurality of types of digital features for each set of time-series data in the plurality of sets; for each model of a plurality of models, calculating a posterior probability that said model is an optimal model for detecting whether the subject is experiencing the event given the received training data, wherein each model of the plurality of models accepts a different subset of the plurality of types of digital features as an input; selecting a narrowed subset of the plurality of types of digital features based on the calculated probabilities; and training a machine-learning model using the narrowed subset of types of digital features to detect whether the subject is experiencing the event.

Among other advantages, the methods and/or systems described herein provide a way to automatically detect, without user input or intervention, when a subject is experiencing an event. A subject may “experience” an event either because the subject is initiating a specific action or activity, or because an event that the subject did not initiate is happening to the subject. Examples of “events” include an eating event in which the subject ingests food or drink, as well as a scratching event, in which the subject scratches a portion of his or her body. Such methods and/or systems may be deployed on a mobile device that detects events. For example, in the case of detecting eating events, such detected eating behavior may be used to remind persons with diabetes to take their insulin bolus doses, and/or to maintain a log of eating episodes. In the case of scratching events, such detected scratching behavior may be used to update a log of scratching behavior, which may in turn be analyzed (e.g., by a HCP or clinical researcher) a presence, severity, and/or progression of a skin condition. Another advantage of the presently disclosed methods and/or systems is that they may be implemented using computing devices with relatively modest and/or limited computing resources (e.g., processing speed, power, and/or memory). As a result, the disclosed methods and/or systems may, in some embodiments, be deployed primarily or solely on a user's mobile device in communication with a user's actigraphy device. In such embodiments, the mobile device need not send data to a remote or cloud server for processing in order to detect events based on actigraphy data. In some embodiments, the disclosed methods and/or systems may be deployed solely on a user's actigraphy device (e.g., smartwatch), without requiring that the actigraphy device be in communication with an associated mobile device (e.g., smartphone). Other advantages will be recognized by those of ordinary skill in the art.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 depicts a system for detecting whether a human subject is experiencing an event, according to some embodiments.

FIG. 2 depicts an exemplary process for detecting the event that may be implemented by the system, according to some embodiments.

FIGS. 3A-3G depicts an example that illustrates how to compute a set of birth and death coordinates for a plurality of topological features in an exemplary set of time-series data, according to some embodiments.

FIG. 4 depicts a process that uses a Bayesian probabilistic approach for selecting a set of digital features to use in detecting whether the subject is experiencing the event, according to some embodiments.

FIG. 5 depicts an example that illustrates calculating posterior inclusion probabilities (PIPs) for evaluated digital features, according to some embodiments.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

A subject's movement may be detected and/or measured using actigraphy devices worn on the subject's body. One example of such an actigraphy device is a wrist-mounted actigraphy device incorporating accelerometer and/or movement sensors. Such actigraphy devices may be designed to be small and non-intrusive such that they may be worn by the subject as he/she goes about his/her daily life, and collect data over a relatively long period of time (e.g., a day, or multiple days).

Time-series movement data collected or derived from such actigraphy devices may be analyzed to detect whether the subject is engaging in certain actions or activities. This analysis and detection of actions/activities may be done without requiring any user input from the subject, or requiring only minimal user input from the subject. This may be useful for minimizing the burden on users to automatically log certain actions or activities.

For example, time-series movement data may be used to automatically detect whether the subject is eating. Automatic eating behavior detection has wide ranges of practical clinical applications. For instance, it is often important for persons with diabetes to inject insulin on a timely basis before and after ingesting food. However, persons with diabetes often miss their bolus doses because they simply forget to take their insulin after a meal. One possible way to reduce the number of missed bolus doses is to use a real-time algorithm deployable on a mobile device that detects eating behavior and reminds patients to take their doses. Passively collected data from wearable sensors such as accelerometers and/or gyroscopes may provide valuable information and allow a user's mobile device(s) to automatically prompt the user to take insulin after ingesting food.

Time-series movement data may also be used to automatically detect whether the subject is scratching. Automatic scratching detection may be useful to detect the presence and/or severity of skin conditions (e.g., atopic dermatitis) that the subject may be suffering from. Tracking of scratching behavior over a prolonged monitoring period (e.g., over a period of days or weeks) may also be useful to assess the progression of such skin conditions. Tracking the progression of skin conditions, in turn, has a wide range of beneficial applications. For example, the ability to track the progression of skin conditions may be useful in clinical trials for evaluating the efficacy of a certain intervention in alleviating or mitigating the skin condition. Such an ability to track the progression of skin conditions may also be useful in clinical practice by giving health care providers (HCPs) the ability to monitor their patients more closely, and determine which treatments are or are not effective. However, manually maintaining a log of scratching behavior is burdensome to users. Scratching behavior may be difficult to quantify, and if scratching occurs at night while users are asleep, users may not be able to accurately log the extent of scratching behavior. Passively collected data from wearable sensors such as accelerometers and/or gyroscopes may provide valuable information to allow a user's mobile device to automatically log the user's scratching behavior.

For ease of explication, the description herein focuses on the use of time-series movement data to detect eating (i.e., detect an eating event) and/or scratching behavior (i.e., detect a scratching event). However, the digital features and techniques presented herein may be generalized to detect any desired user action or activity, or any event which may be experienced by the subject. For example, the techniques presented herein may be used to detect whether the user is walking, running, and/or exercising. The techniques presented herein may also be used to detect and/or infer insights about the way in which the user is executing a certain action or activity. For example, the techniques presented herein may be used not simply to detect the presence of scratching, but also to assess characteristics of the scratching behavior, e.g., its duration, its vigorousness, etc.

Such a detection algorithm would ideally (but not necessarily) run in real-time, such that results may be presented to the user almost immediately upon occurrence of the action or activity. It would also be advantageous to make such a real-time algorithm as computationally efficient as possible such that it requires less computational resources and/or time to run. A lightweight algorithm may be deployable entirely or primarily on a user's mobile device (e.g., smartphone, smartwatch, and/or laptop computer) without requiring that data be sent over a network to a remote server for processing. Such an algorithm may be easier to use in geographic locations where reliable network or Internet access is difficult, may be cheaper to implement at scale, and provide users with faster results. As a result, it would be desirable to simplify such an algorithm to make it easier or faster to run. The devices, systems, and methods disclosed herein are directed at addressing these issues, among others.

The term “logic,” “control logic,” “application,” “process,” “method,” “algorithm,” and “instructions” as used herein may include software and/or firmware executing on one or more programmable processors, application-specific integrated circuits (ASICs), field-programmable gate arrays (FPGAs), digital signal processors (DSPs), hardwired logic, or combinations thereof. Therefore, in accordance with the embodiments, various logic may be implemented in any appropriate fashion and would remain in accordance with the embodiments herein disclosed.

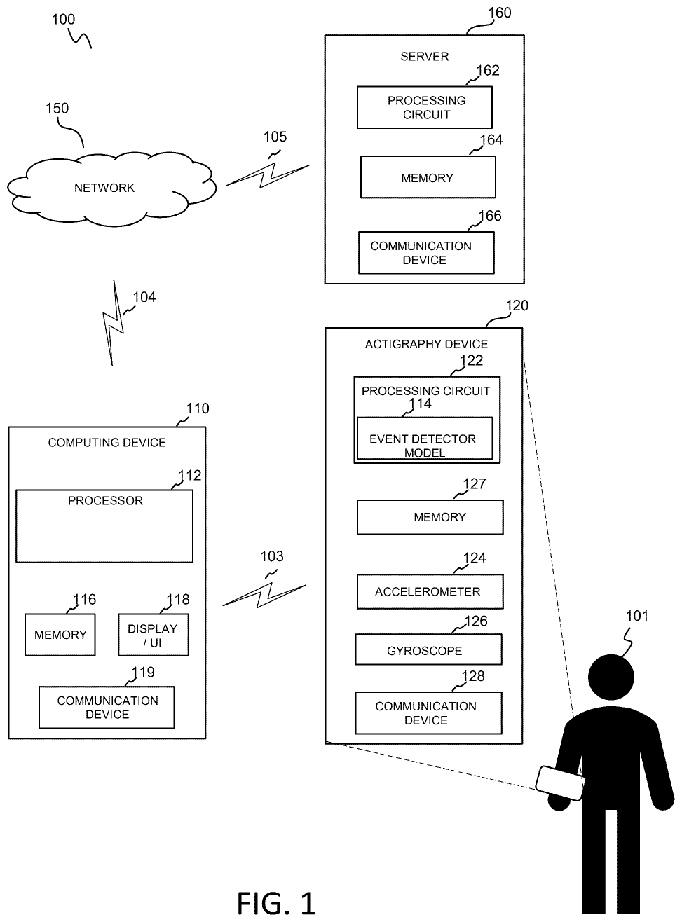

FIG. 1 depicts a system 100 for detecting an event. An event may include an eating event, in which a human subject 101 ingests food, or in which a human subject 101 ingests food or drink. Alternatively, an event may include a scratching event, in which a human subject 101 scratches a portion of his/her body. In yet other embodiments, an event may include other activities or actions exhibited by the human subject 101. System 100 includes a wearable actigraphy device 120 worn on a wrist of the subject 101. The actigraphy device 120 may optionally be in wireless communication with a computing device 110. Computing device 110 may also be in communication with a server 160 via a network 150.

Computing device 110 illustratively includes a mobile device, such as smartphone. Alternatively, any suitable computing device may be used, including but not limited to a laptop, desktop, tablet, or server computer, for example. Computing device 110 includes processor 112, memory 116, display/user-interface (UI) 118, and communication device 119.

Processor 112 includes at least one processor that executes software and/or firmware stored in memory 116 of computing device 110. The software/firmware code contains instructions that, when executed by processor 112, cause processor 112 to perform the functions described herein. Such instructions illustratively include event detector model 114 operative to implement the functionality described in further detail below. Memory 116 is any suitable computer readable medium that is accessible by processor 112. Memory 116 may be a single storage device or multiple storage devices, may be located internally or externally to processor 112, and may include both volatile and non-volatile media. Exemplary memory 116 includes random-access memory (RAM), read-only memory (ROM), electrically erasable programmable ROM (EEPROM), flash memory, a magnetic storage device, optical disk storage, or any other suitable medium which is configured to store data and which is accessible by processor 112.

Computing device 110 includes a display/user interface 118 in communication with processor 112 and operative to provide user input data to the system and to receive and display data, information, and prompts generated by the system. User interface 118 includes at least one input device for receiving user input and providing the user input to the system. In the illustrated embodiment, user interface 118 is a graphical user interface (GUI) including a touchscreen display operative to display data and receive user inputs. The touchscreen display allows the user to interact with presented information, menus, buttons, and other data to receive information from the system and to provide user input into the system. Alternatively, a keyboard, keypad, microphone, mouse pointer, or other suitable user input device may be provided.

Computing device 110 further includes communication device 119 that allows computing device 110 to establish wired and/or wireless communication links with other devices. Communication device 119 may comprise one or more wireless antennas and/or signal processing circuits for sending and receiving wireless communications, and/or one or more ports for receiving physical wires for sending and receiving data. Using communication device 119, computing device 110 may establish one or more short-range communication links, including one or more of communication links 103 with actigraphy device 120. Such short-range communication links may utilize any known wired or wireless communication technology or protocol, including without limitation radio frequency communications (e.g., Wi-Fi, Bluetooth, Bluetooth Low Energy (BLE), Near Field Communications (NFC), RFID, and the like), infrared transmissions, microwave transmissions, and lightwave transmissions. Such short-range communication links may be either uni-directional links (e.g., data flows solely from actigraphy device 120 to computing device 110), or bi-directional links (e.g., data flows both ways). Communication device 119 may also allow computing device 110 to establish a long-range communication link with a server 160 via a network 150, and communication links 104 and 105. The server 160 may be located remote from computing device 110, e.g., in another building, in another city, or even in another country or continent. Network 150 may comprise any cellular or data network adapted to relay information from computing device 110 to and/or from server 160, potentially via one or more intermediate nodes or switches. Examples of suitable networks 150 include a cellular network, a metropolitan area network (MAN), a wide area network (WAN), and the Internet.

Actigraphy device 120 illustratively includes any sensor device worn on a body of human subject 101 and to record data regarding the movements and/or physiological properties of subject 101. Actigraphy device 120 may include a plurality of sensors for detecting and/or measuring such data, such as an accelerometer 124 and a gyroscope 126. Each of accelerometer 124 and gyroscope 126 may be configured to measure and/or record accelerometer and gyroscope data in one, two, or three dimensions. Although not shown, actigraphy device 120 may also include alternative or additional sensors, such as an electromyogram (EMG) sensor, a mechanomyogram sensor (MMG) sensor, a sound or vibration sensor, a light sensor, or the like. Actigraphy device 120 may also comprise a processing circuit 122, which may include any processing circuit that receives and processes data signals, and which outputs results in the form of one or more electrical signals as a result. Processing circuit 122 may include a processor (similar to processor 112), an Application Specific Integrated Circuit (ASIC), field-programmable gate array (FPGAs), digital signal processors (DSPs), hardwired logic, or combinations thereof. Actigraphy device may also include a memory 127, which may include any of the possible types of memory previously described. Actigraphy device also includes a communication device 128, which allows actigraphy device 120 to communicate with computing device 110 via communication link 103, and relay measured/recorded data to computing device 110. As illustrated in FIG. 1, actigraphy device 120 may be a wrist-mounted actigraphy device worn on a dominant wrist of the subject 101. In addition, actigraphy device 120 may be worn on a non-dominant wrist of the subject 101. Examples of suitable wrist-mounted wearable sensors include the Apple Watch®, the FitBit®, and the like. In addition, actigraphy device 120 may be worn on other portions of subject 101's body, such as on the subject 101's torso, neck, arm, or shoulder.

Server 160 illustratively includes any computing device configured to receive and process information from either actigraphy device 120 or from computing device 110, either directly or via network 150. Optionally, server 160 may also be configured to send responses, notifications, or instructions to computing device 110, actigraphy device 120, or another device in response to said information. Server 160 includes processing circuit 162, memory 164, and communication device 166. Processing circuit 162 may include any of the possible types of processing circuits previously described. Processing circuit 162 may execute software and/or firmware stored in memory 164 of server 160. The software/firmware code contains instructions that, when executed by processing circuit 162 to perform the functions described herein. Memory 164 may also be configured to store information regarding one or more persons with diabetes, such as biographical information and/or medical information (e.g., insulin dosing records, medical history, logs of subject scratching behavior, and the like). Information received from or sent to computing device 110 may also be stored in memory 164. Memory 164 may include any of the possible types of memory previously described. Communication device 166 allows server 160 to communicate with computing device 110 via communication link 105, network 150, and communication link 104.

In some embodiments, system 100 may be modified by omitting one or more components. For example, if actigraphy device 120 is equipped with a long-range communication interface, actigraphy device 120 may communicate directly with server 160 via network 150, bypassing computing device 110 completely. In some embodiments, server 160 may be omitted and the methods and algorithms disclosed herein may be implemented solely on computing device 110, or solely on computing device 110 in conjunction with actigraphy device 120. In some embodiments, both computing device 110 and server 160 may be omitted, and the methods and algorithms disclosed herein may be implemented solely on actigraphy device 120, without requiring that device 120 be in communication with any other device. In other embodiments, system 100 may be modified by adding components. For example, server 160 may be configured as a plurality of networked servers 160 that cooperate to process information. Such a configuration of networked servers may be referred to as a “cloud” of servers that perform the functions described herein. The server(s) 160 may communicate with multiple computing devices 110 via network 150. In yet other embodiments, there may be multiple wearable actigraphy devices 120 worn on the body of subject 101, each collecting the same or different types of data.

System 100 has been described herein as implementing an event detector model 114 within processor 122 of actigraphy device 120. Event detector model 114 may be a meal detector model, a scratching detector model, or a model configured to detect some other type of subject action or activity. As described in further detail below, event detector model 114 is configured to detect whether certain user actions or activities (e.g., meals, scratching) based on data measured and/or recorded by wearable actigraphy device 120. Event detector model 114 may comprise software instructions and/or logic that is stored in memory 127 and which is executed by processor 112 to implement the functionality described herein. However, detector 114 may take other forms in other embodiments. For example, the functionality performed by detector model 114 may be implemented at least partially by dedicated hardware and/or firmware, such as a separate and dedicated processor and/or processing circuit. In some embodiments, the functionality performed by detector 114 may also be implemented wholly or partially on computing device 110, and/or server 160.

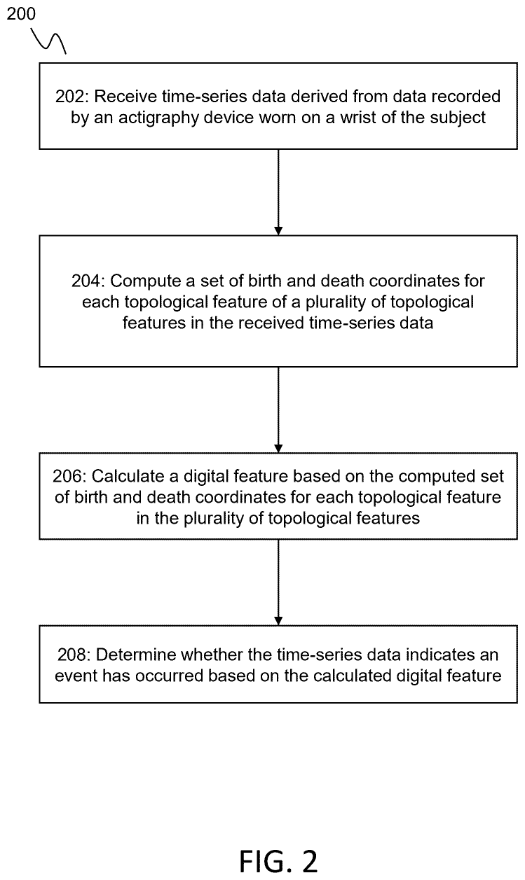

FIG. 2 depicts an exemplary process 200 for detecting events, according to some embodiments. For simplicity of explication, the steps described in FIG. 2 are described below as being implemented on processing circuit 122 of actigraphy device 120, e.g., as part of event detector model 114. However, it should be understood that any of the steps described below with respect to FIG. 2 may be implemented on either computing device 110 or server 160. In some embodiments, some or all of the aforementioned devices may cooperate to implement process 200, e.g., one device may perform some of the steps, while another device performs other steps, or one device may perform some of the steps with the help of intermediate computations and/or data provided by other device(s).

Process 200 begins at step 202, where processing circuit 122 at actigraphy device 120 receives time-series data derived from raw data recorded by actigraphy device 120 worn on a wrist of the subject. In some embodiments, the raw data recorded by the actigraphy device may comprise accelerometer data (e.g., in three dimensions, such as along x, y, and z axis) and gyroscope data (e.g., also in three dimensions, such as along x, y, and z axis). For ease of explication, let ax, ay, and a_{z} denote the accelerometer data along the x-axis, y-axis, and z-axis respectively, and let gₓ, g_{y}, and g_{z} denote the gyroscope data along the x-axis, y-axis, and z-axis respectively. Such raw data may be recorded using any suitable sampling rate. In one exemplary embodiment, a sampling rate of 50 Hz may be used, though faster or slower sampling rates may also be used. As previously mentioned, the actigraphy device may comprise a wearable sensor worn on a dominant wrist or non-dominant wrist of the subject, though other types of actigraphy devices may also be used. For detecting events that are primarily done by one dominant hand (e.g., for eating events that are primarily done using the subject's dominant hand), the actigraphy device may be configured to be worn on the subject's dominant wrist. The recorded raw data may comprise data recorded over a pre-determined time period, such as a one-minute period, a two-minute period, a five-minute period, a ten-minute period, or another suitable time period. So, to summarize, in one exemplary embodiment, the raw recorded data may comprise six separate data streams: aₓ, a_{y}, a_{z}, gₓ, g_{y}, and g_{z} data, each recorded over a concurrent one-minute period using a sampling rate of 50 Hz (i.e., each data stream of the six data streams has 3,000 separate datapoints, for a total of 18,000 datapoints, all for the same one-minute period).

The received time-series data may be derived from the raw recorded data in various ways. In some embodiments, the received time-series data may be the same as the raw recorded data, i.e., the time-series data may also be aₓ, a_{y}, a_{z}, gₓ, g_{y}, and g_{z}. In other embodiments, the received time-series data may be calculated according to one, some, or all of the recorded data. For example, the received time-series data may be the ratio of the sums of the raw recorded data

$$( e . g . , \frac{\vert {g}_{x} \vert + \vert {g}_{y} \vert + \vert {g}_{z} \vert}{\vert {a}_{x} \vert + \vert {a}_{y} \vert + \vert {a}_{z} \vert}$$

$$and / or$$

$$\frac{\vert {a}_{x} \vert + \vert {a}_{y} \vert + \vert {a}_{z} \vert}{\vert {g}_{x} \vert + \vert {g}_{y} \vert + \vert {g}_{z} \vert} ) ,$$

the angles of accelerometer data

$$( e . g . , \frac{{a}_{x}}{\sqrt{{a}_{x}^{2} + {a}_{y}^{2} + {a}_{z}^{2}}} ,$$

$$\frac{{a}_{y}}{\sqrt{{a}_{x}^{2} + {a}_{y}^{2} + {a}_{z}^{2}}} ,$$

$$and / or$$

$$\frac{{a}_{z}}{\sqrt{{a}_{x}^{2} + {a}_{y}^{2} + {a}_{z}^{2}}} ) ,$$

and/or the angles of gyroscope data

$$( e . g . , \frac{{g}_{x}}{\sqrt{{g}_{x}^{2} + {g}_{y}^{2} + {g}_{z}^{2}}} ,$$

$$\frac{{g}_{y}}{\sqrt{{g}_{x}^{2} + {g}_{y}^{2} + {g}_{z}^{2}}} ,$$

$$and / or$$

$$\frac{{g}_{z}}{\sqrt{{g}_{x}^{2} + {g}_{y}^{2} + {g}_{z}^{2}}} ) .$$

Other suitable ways of deriving the received time-series data from the raw recorded data may also be used. The received time-series data may be derived from the raw recorded data by processor 122 of actigraphy device 120, or by processor 112 of computing device 110.

At step 204, computing device 110 computes a set of birth and death coordinates for each topological feature of a plurality of topological features in the received time-series data. Such coordinates may be calculated using techniques drawn from the mathematical field of topological data analysis. The set of birth and death coordinates corresponding to each topological feature may also be referred to as a “persistence diagram” in the field of topological data analysis. Topological features are derived from a sub-level set associated with a filtration threshold. A sub-level set fₜ⁻ for time-series data y(x) associated with filtration threshold t is defined as the set of times x for which y(x) is less than or equal to threshold. In mathematical notation, a sublevel set fₜ⁻ associated with filtration threshold t may be expressed according to equation 1:

$${f}_{t}^{-} = \{ x \vert y ( x ) \leq t \} \qquad Equation 1$$

A topological feature within sublevel set fₜ⁻ is a contiguous segment of time (x) within fₜ⁻ that is not interrupted by any gaps in time. A sublevel set fₜ⁻ may have zero, one, or more topological features.

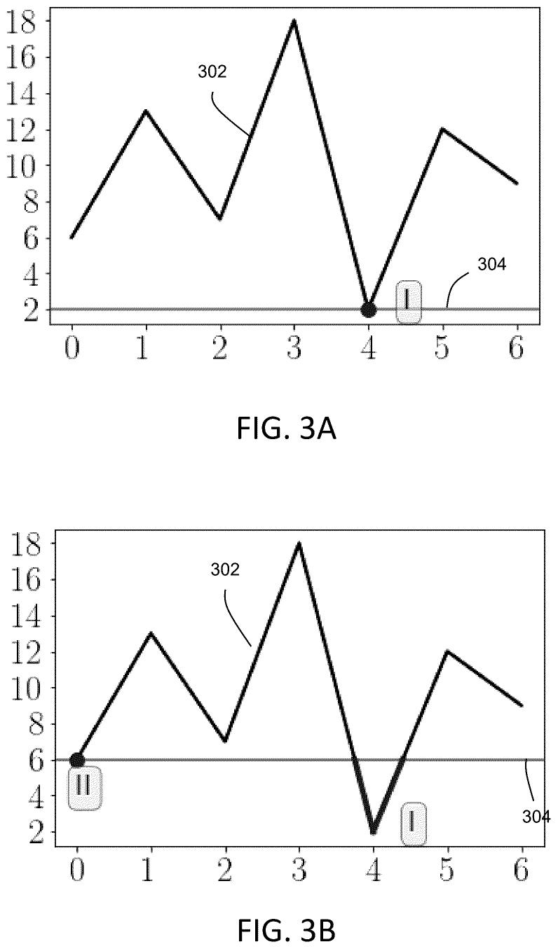

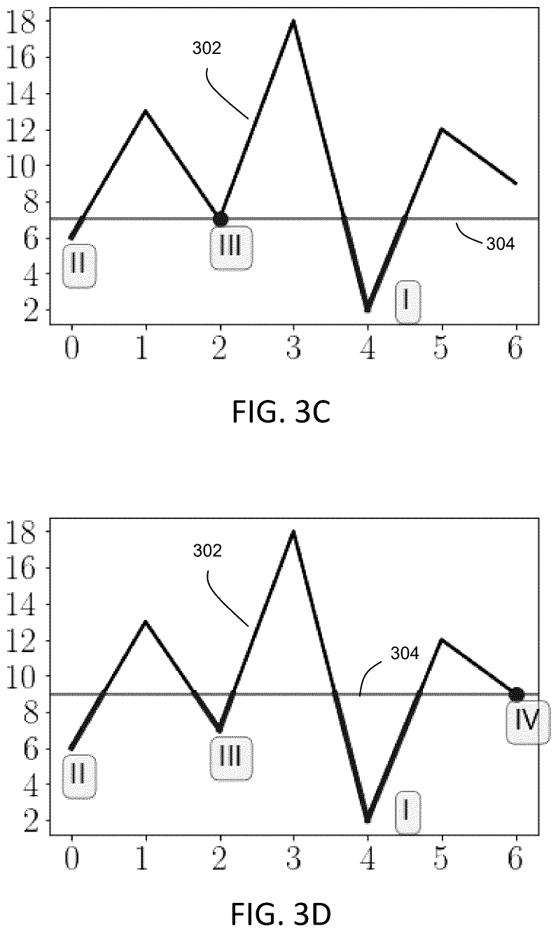

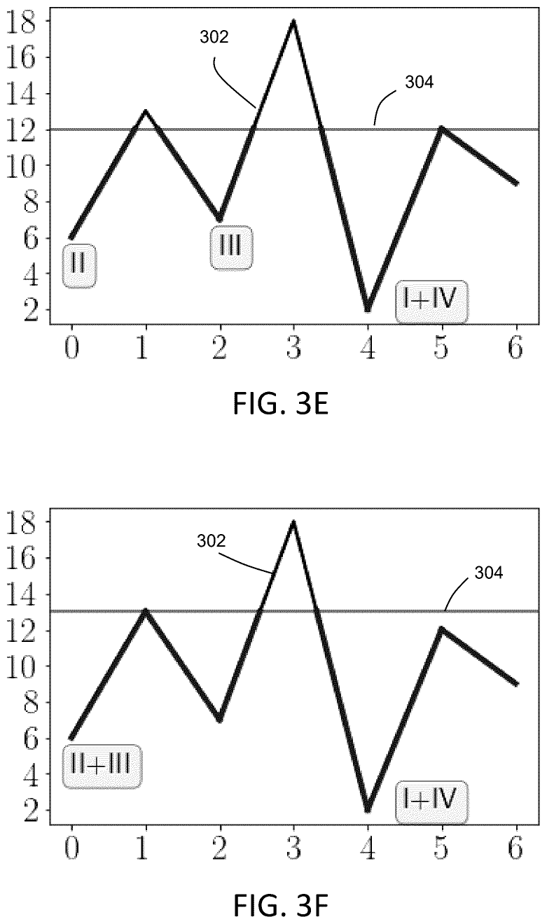

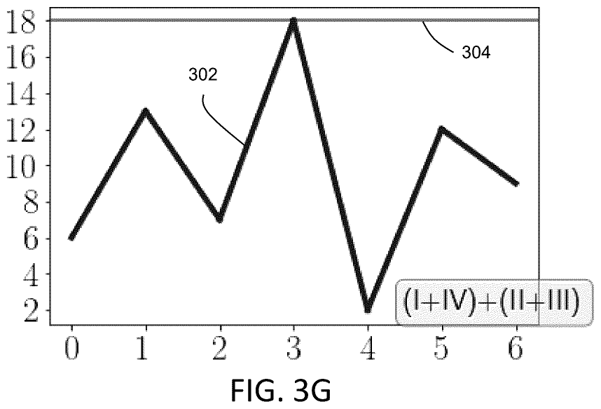

FIGS. 3A-3G illustrate graphically how to compute a set of birth and death coordinates for a plurality of topological features in an exemplary set of time-series data 302. Exemplary time-series data 302 may be any of the processed time-series data previously discussed as part of step 202. For clarity, the time-series data 302 presented in FIGS. 3A-3G are illustrative values only, and not the result of any actual experiment or simulation. In each of FIGS. 3A-3D, the x-axis represents time while the y-axis represents the value or magnitude of time-series data 302. To uncover topological features, time-series data 302 is analyzed while increasing the filtration threshold t from a minimum value corresponding to a minimum value of time-series data 302 to a maximum value corresponding to a maximum value of time-series data 302. Each figure in FIGS. 3A-3G is associated with a different filtration threshold/(marked 304 in the figures). For instance, FIG. 3A has a filtration threshold 304 of 2, FIG. 3B has a filtration threshold 304 of 6, FIG. 3C has a filtration threshold 304 of 7, FIG. 3D has a filtration threshold 304 of 9, FIG. 3E has a filtration threshold 304 of 12, FIG. 3F has a filtration threshold 304 of 13, and FIG. 3G has a filtration threshold of 18.

In this simplified example, FIG. 3A (which has a filtration threshold of 2) has one topological feature, denoted by roman numeral “I”. This feature “I” is the only continuous segment of time in the sublevel set f₀⁻. FIG. 3B (which has a filtration threshold of 6) has two topological features, denoted by roman numerals “I” and “II”. Again, each feature is associated with a continuous segment of time in the sublevel set f_{0.5}⁻, i.e., each feature is associated with a continuous segment of time for which the time-series data 302 is below the filtration threshold 304. Similarly, FIG. 3C (which has a filtration threshold of 7) has three topological features, denoted “I”, “II”, and “III”, FIG. 3D (which has a filtration threshold of 9) has four topological features, denoted “I”, “II”, “III”, and “IV”, FIG. 3E (which has a filtration threshold of 12) has three topological features, denoted “I+IV”, “II”, and “III”, FIG. 3F has two topological features, denoted “I+IV” and “II+III”, and FIG. 3G has one topological feature, denoted “(I+IV)+ (II+III).”

As the filtration threshold 304 increases, topological features may be expected to change. Topological features that overlap in time are considered the “same” topological feature. So for instance, topological feature “I” in FIGS. 3A and 3B are considered the “same” topological feature because these features overlap in time even as filtration threshold 304 increases from 2 to 6. As filtration threshold 304 increases, some topological features may be expected to merge. For instance, as depicted in FIG. 3E, as the filtration threshold 304 increases from 9 to 12, topological feature “I” (which first appears in FIG. 3A) and topological feature “IV” (which first appears in FIG. 3D) merge. In such situations, the topological feature that has an earlier “birth” (as described below) is considered to survive, while the topological feature that has a later “birth” is considered to expire. For that reason, although FIG. 3E labels the merged topological feature “I+IV” for clarity, the merged feature in FIG. 3E may alternatively be labeled simply feature “I”, since feature “I” has an earlier birth than feature “IV”. Similarly, as depicted in FIG. 3F, as the filtration threshold 304 increases from 12 to 13, topological feature “II” and topological feature “III” merge. Since topological feature “II” (which first appears in FIG. 3B, when the filtration threshold 304 is equal to 6) has an earlier “birth” than feature “III” (which first appears in FIG. 3C, when the filtration threshold 304 is equal to 7), feature “II” is considered to survive while feature “III” is considered to expire. For that reason, although FIG. 3F labels the merged topological feature “II+III” for clarity, the merged feature in FIG. 3F may alternatively be labeled simply feature “II”. Finally, as depicted in FIG. 3G, as the filtration threshold 304 increases from 13 to 18, topological feature “I+IV” (or simply, feature “I” as previously described) and topological feature “II+III” (or simply feature “II” as previously described) merge. Since topological feature “I” (which first appears in FIG. 3A, when filtration threshold 304 equaled 2) has an earlier birth than topological feature “II” (which first appears in FIG. 3B, when filtration threshold 304 equaled 6), feature “I” is considered to survive while feature “II” is considered to expire. For that reason, although FIG. 3G labels the merged topological feature “(I+IV)+ (II+III)” for clarity, the merged feature in FIG. 3G may alternatively be labeled simply feature “I”.

The “birth” coordinate for a particular topological feature is the filtration threshold level 1 at which that particular topological feature comes into existence as the filtration threshold level is increased from the minimum value of y(x) to the maximum value of y(x). Similarly, the “death” coordinate for a particular topological feature is the filtration threshold level/at which that particular topological feature expires. A particular topological feature is considered to “expire” when it merges with another topological feature having an earlier birth coordinate, as discussed above.

Turning to the examples in FIGS. 3A-3G, it can be seen that the exemplary time-series data 302 has four topological features. Topological feature “III” comes into existence when filtration threshold level 304 equals 7, hence feature “III” is considered to have a birth coordinate of 7. Feature “III” expires when filtration threshold level 304 increases to 13, at which point feature “III” merges with feature “II”. Hence, topological feature “III” is considered to have a death coordinate of 13. Expressed together, topological feature “III” is considered to have a birth-death coordinate, expressed in the form [birth coordinate, death coordinate], of [7, 13].

Topological feature “IV” similarly comes into existence when filtration threshold level 304=9, hence feature “IV” is considered to have a birth coordinate of 9. Feature “IV” expires when filtration threshold level 304 equals 12, at which point feature “IV” merges with feature “I”. Hence, topological feature “IV” is considered to have a death coordinate of 12. Expressed together, topological feature “IV” is considered to have a birth-death coordinate of [9, 12].

Topological feature “II” comes into existence when filtration threshold 304 equals 6, hence feature “II” is considered to have a birth coordinate of 6. Feature “II” expires when filtration threshold level 304 equals 18, at which point feature “II” merges with feature “I”. Hence, topological feature “II” is considered to have a death coordinate of 18. Expressed together, topological feature “II” is considered to have a birth-death coordinate of [6,18].

Topological feature “I” comes into existence when filtration threshold 304 equals 2, hence feature “I” is considered to have a birth coordinate of 2. Feature “I” does not merge with any other topological feature even when filtration threshold level 304 is increased to the maximum value of time-series 302, i.e., 18. Hence, feature “I” is considered to expire at the maximum value for filtration threshold 304, which is 18. Expressed together, feature “I” is considered to have a birth-death coordinate of [2, 18].

In the exemplary scenario depicted in FIGS. 3A-3G, every time two topological features merge (e.g., when feature “I” merged with feature “IV”, or when feature “II” merged with feature “III”), one of the merging features had an earlier birth coordinate than the other merging feature. It was therefore clear which of the merging features would survive and which of the merging features would expire. In some cases involving different time-series data, however, two features having identical birth coordinates may merge. In such cases, the merging feature that expires and the merging feature that survives may be determined in other ways. For example, the surviving/expiring feature may be randomly determined. Or alternatively, the feature that occurs earlier or later in time may be designated the surviving feature.

Returning to FIG. 2, at step 206, computing device 110 calculates a digital feature based on the computed set of birth and death coordinates (i.e., the persistence diagram) for each topological feature in the plurality of topological features. A digital feature may simply be a value that is derived in some way from the time series data 302. For example, in some embodiments, computing device 110 may use all or some subset of the computed set of birth and death coordinates (i.e., the persistence diagram) as the digital feature. In some embodiments, at step 206, computing device 110 may calculate a lifespan persistence (L) for each respective topological feature in the plurality of topological features by subtracting the birth coordinate for that respective topological feature from the death coordinate for that respective topological feature. Computing device 110 may then calculate a digital feature based on at least one of a mean, a standard deviation, a skewness, a kurtosis, and an entropy of the calculated lifespan persistence (L) across all topological features in the received time-series data. In some embodiments, alternatively or in addition, computing device 110 may calculate a midlife persistence (M) for each respective topological feature in the plurality of topological feature by calculating a mean average of the set of birth and death coordinates for that respective topological feature. Computing device 110 may then calculate a digital feature based on at least one of a mean, a standard deviation, a skewness, a kurtosis, and an entropy of the calculated midlife persistence (M) across all topological features in the received time-series data.

In some embodiments, at step 206, computing device 110 may calculate a digital feature based on a norm of a Gaussian persistence curve, according to Equation 2:

$$‖ {G}_{D} ‖ = {\sum }_{( b , d ) ∈ D} [ ( d - b ) Φ ( \frac{d - b}{\sqrt{2} σ} ) + \sqrt{2} σ ϕ ( \frac{d - b}{\sqrt{2} σ} ) ] \qquad Equation 2$$

where ϕ(x) is the probability density function of a standard normal distribution, Φ(x) is the cumulative density function of a standard normal distribution, b is a birth coordinate, d is a death coordinate, D is the set of all birth and death coordinate pairs (b,d), and σ is the standard deviation of the normal distribution. In some embodiments, σ may be a user-defined parameter. For example, a user may set σ to a value of 1.

At step 208, computing device 110 may determine whether the time-series data indicates an event (e.g., an eating event, a scratching event, or some other type of event) has occurred based on the calculated digital feature. In many embodiments, computing device 110 may make this determination based on a plurality of digital features (i.e., not just one digital feature). Computing device 110 may also make this determination based on other information. In some embodiments, computing device 110 may make this determination using a rules-based, logical approach (e.g., if-then or nested if-then statements). In some embodiments, computing device 110 may make this determination using a trained machine-learning model that accepts as input at least one digital feature calculated from step 206, and outputs a probability and/or a binary prediction regarding whether the event has occurred (e.g., for an eating event, whether the subject is eating or not eating; for a scratching event, whether the subject is scratching), given the inputted digital feature. The machine-learning model may include a neural network model or any other suitable model or models, which will be further described herein.

If a machine-learning model is used in step 208, the machine learning model may be pre-trained using training data. A plurality of sets of training time-series data similar to that discussed above in step 202 (e.g., wherein each set of training time-series data was recorded over a one-minute, two-minute, five-minute, ten-minute, or other time period), in conjunction with ground truth labels indicating whether each set of training time-series data is associated with an event or not, may be provided. For example, training data may be recorded from a plurality of test subjects who are being observed to determine whether they are undertaking a specific targeted subject action or activity may be recorded. If the methods discussed herein are being used to detect eating events, the training data may comprise data regarding whether the subject was eating or not eating at the time the training data was recorded. If the methods discussed herein are being used to detect scratching events, the training data may comprise data regarding whether the subject is scratching or not scratching at the time the training data was recorded. A set of birth and death coordinates for each topological feature may be calculated, i.e., a persistence diagram (similar to step 204), and one or more digital features may be calculated (similar to step 206) for each set of the training time-series data. These calculated digital features, in conjunction with the ground truth event/no-event label, may be used to train the machine-learning model. The machine-learning model may be pre-trained by the same device (e.g., computing device 110) that is using the model to detect events. Alternatively, the machine-learning model may be pre-trained by another device, such as server 160.

The machine learning model may take different forms and utilize any appropriate machine learning techniques. In some embodiments, the machine learning model may employ a gradient boosting machine learning technique, which gives a prediction model in the form of an ensemble of weak prediction models, which may be decision trees. Alternatively, or in addition, the machine learning model may include a neural network model that includes trained weights. Examples of a neural network model may include a convolutional neural network (CNN) or a simple neural network or any variations thereof. A method for training the machine learning model may use any training algorithm or algorithms, such as, for example, gradient descent and/or any other suitable algorithms. The trained machine learning model may be configured to output an indication of whether the subject is experiencing a specific event or not, e.g., a binary indication (event vs. no event) or a probability that the subject is experiencing the event, based on the calculated digital features.

The process in FIG. 2 may be modified by altering, rearranging, deleting, and/or substituting any of process steps 202, 204, 206, and/or 208. For example, in some embodiments, instead of calculating digital features according to steps 204 and 206, other types of digital features may be derived in alternate ways from the time-series data received at step 202. Examples of other suitable digital features derived from a received time-series data {sᵢ}ᵢ₌₁^{N} where N indicates the number of data points, includes:
The mean, standard deviation, skewness, kurtosis, and/or entropy of sᵢ;
The range of sᵢ, i.e., max(sᵢ)−min(sᵢ);
The energy of sᵢ, e.g.,

$$\frac{1}{N} {\sum }_{i = 1}^{N} {s}_{i}^{2} ;$$

The wavelength of si, e.g., Σᵢ₌₁^{N-1}|sᵢ₊₁−sᵢ|
The entropy of the frequency of sᵢ; and
The zero crossing for sᵢ.

Any or all of the aforementioned digital features may also be used as part of step 208 to determine whether the time-series data indicates an event, whether alternatively or in addition to the digital features previously described in steps 204 and 206. Since many of the aforementioned digital features may be computed relatively easily for time-series data of appropriate length (e.g., in one, five, or ten minute increments), the aforementioned digital features may be computed using a mobile device possessing only modest or limited computing resources (e.g., computational power and/or memory). This allows process 200 to be implemented primarily or solely on an actigraphy device 120 (e.g., a smartwatch) or computing device 120 (e.g., a smartphone), without requiring that data be sent to a remote server or to a cloud computing resource for computation.

In some cases, in order to further reduce the computational burden on the computational device implementing process 200, it may be desirable to use only a subset of the aforementioned digital features to detect eating events. For example, the aforementioned machine-learning model may be trained using only a selected subset of the aforementioned digital features-then in the testing phase, the model may be provided data corresponding only to the selected subset of digital features. In this way, the computational time and/or resources required to implement both the testing and use of the machine-learning model may be reduced.

In such embodiments, it may be useful to determine which digital features of the aforementioned digital features would be best to use for detecting an event. This determination of which digital features to use may be done ahead of time in an offline manner, before training and use of the machine-learning model. Ideally, the selected digital features would be the digital features that contribute the most to the accuracy and reliability of the eventual trained model.

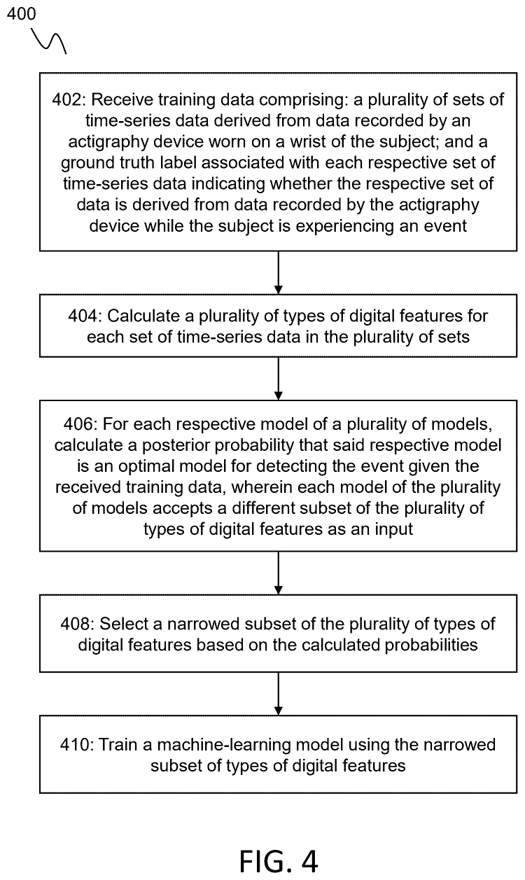

FIG. 4 depicts a process 400 that uses an approach based on Bayesian probability for selecting a set of digital features to use in detecting an event, according to some embodiments. For simplicity of explication, the steps described in FIG. 4 are described below as being implemented on server 160. Since selection of digital features and training of a machine-learning model generally takes a significant amount of time and/or computing resources, such steps are generally executed on server 160 in an offline manner. However, it should be understood that any of the steps described below with respect to FIG. 4 may be implemented on either actigraphy device 120 or computing device 110. In some embodiments, some or all of the aforementioned devices may cooperate to implement process 400, e.g., one device may perform some of the steps, while another device performs other steps, or one device may perform some of the steps with the help or intermediate computations and/or data provided by other device(s).

Process 400 begins at step 402, where server 160 receives training data. The training data may comprise a plurality of sets of time-series data derived from data recorded by an actigraphy device worn on a wrist of a subject. The time-series data may be similar to the time-series data described above with respect to step 202 of process 200. In addition, the training data may comprise a ground truth label associated with each respective set of time-series data indicating whether the respective set of data is derived from data recorded by the actigraphy device while the subject is experiencing an event (e.g., a label indicating whether the respective set of data is associated with an eating event or not, or whether the respective set of data is associated with a scratching event or not). In some embodiments, this ground truth label may be a binary label indicating that the event either occurred or did not occur.

At step 404, server 160 calculates a plurality of types of digital features for each set of time-series data in the plurality of sets. The types of digital features calculated at this step 404 may include any of the types of digital features discussed previously.

At step 406, server 160 calculates a posterior probability for each model of a plurality of models. A model may comprise a potential algorithm (e.g., a trained machine-learning model) that accepts as input all or a subset of the digital features calculated at step 404, and outputs an indication whether the subject is experiencing the event or not (e.g., a binary output indicating that the event either occurred or did not occur). Each model evaluated by server 160 at step 406 accepts a different subset of the plurality of types of digital features as input.

If server 160 calculates n types of digital features at step 404, then the number of possible combinations of digital features, and therefore the number of models that could be evaluated at step 406, is 2ⁿ. Since the number of models that could be evaluated grows exponentially with the number n of digital features, it may be time- and/or resource-intensive to explore all possible models at step 406 even for a modest number n of types of digital features. In some embodiments, in order to reduce time and/or computational resource requirements, server 160 may calculate a posterior probability for only some “explored” models out of all possible models that could be constructed using the digital features calculated at step 404. The posterior probability calculated by server 160 for an explored model corresponds to a posterior probability that said respective model being evaluated is an optimal model for detecting the targeted event given the received training data. As used herein, an explored model may be considered an “optimal” model if the respective explored model's predictions for whether the subject is experiencing the event or not matches the ground truth labels with an accuracy that exceeds or matches (within a certain pre-defined allowance threshold) the accuracy of any other possible model that may be constructed using any subset of the digital features calculated at step 404.

Below is an abbreviated explanation of one exemplary method by which server 160 may calculate such a posterior probability. Full details regarding how this posterior probability may be calculated may be found at Nikooienejad et al., “Bayesian variable selection for binary outcomes in high-dimensional genomic studies using non-local priors,” Bioinformatics, 32 (9), 2016, 1338-1345 (referred to herein as “Nikooienejad 2016”), the full contents of which are hereby incorporated by reference.

Let yₙ=(y₁, . . . , yₙ)^{T} denote a vector of training data comprised of independent binary observations. n is the number of observations, i.e., the number of sets of time-series data received by server 160 at step 402. An individual element yᵢ is a 1 if the ground truth label for the i′th set of time-series data indicates the i′th set of data is associated with an event (e.g., an eating event or a scratching event); otherwise, yᵢ is a 0.

Let p represent the number of types of digital features calculated by server 160 at step 404. For ease of identification, the types of digital features calculated at step 504 may be indexed 1 through p.

Let βₖ be a p×1 regression vector corresponding to a model K. We denote a model K={k₁, . . . , kj} where (1≤k₁< . . . <kⱼ≤p), and where k₁ . . . kⱼ denote the index of the digital features that are accepted as input by model K. Since these digital features are accepted as input, it is assumed that βₖ₁ (i.e., the k₁′th entry of βₖ)≠0, . . . , βₖⱼ (i.e., the kⱼ′th entry of βₖ)≠0, and all other elements of βₖ are 0.

The design matrix corresponding to model K is denoted by Xₖ, which has k columns, wherein each column represents a digital feature accepted as input by model K. We assume that each column of Xₖ has been standardized, such that each column has a mean of 0 and a variance of 1. The i′th row of Xₖ is denoted Xᵢₖ.

We further assume that each individual element yᵢ of the training data yₙ is distributed according to:

$${y}_{i} \vert {β}_{k} \sim Bernoulli [ \frac{exp ( {x}_{ik}^{′} {β}_{k} )}{1 + exp ( {x}_{ik}^{′} {β}_{k} )} ] \qquad Equation 3$$

Under prior constraints on the model space and the assumption of non-local prior density constraints on the regression parameter βₖ, the posterior probability of model K can then be thought of as the probability that model K (having the probability distribution set forth in Equation 3 above) is the model that generated the training data, given that the training data that was actually observed is provided by yₙ.

From Bayes theorem, the posterior probability of a particular model j being the optimal model out of the set of possible models J is specified as:

$$p ( j \vert {y}_{n} ) = \frac{p ( j ) {m}_{j} ( {y}_{n} )}{{\sum }_{κ ∈ J} p ( k ) {m}_{k} ( {y}_{n} )} , \qquad Equation 4$$

$$where$$

$${m}_{k} ( {y}_{n} ) = \int π ( {y}_{n} \vert {β}_{k} ) π ( {β}_{k} ) d {β}_{k} \qquad Equation 5$$

The probability distribution π(yₙ|βₖ) for a particular βₖ is the likelihood of observing training data yₙ given model K with regression coefficients βₖ. and may be calculated according to Equation 3 above. However, calculating the posterior probability for the model j still requires calculating a prior density of regression coefficients π(βₖ) as well as the prior model probabilities for the model K, i.e., p(k).

The prior density π(βₖ) may be calculated using non-local priors by specifying two parameters: τ and r. Specifically, π(βₖ) may be calculated as:

$$π ( {β}_{k} \vert τ , r ) = \frac{{τ}^{rk / 2}}{{Γ ( \frac{r}{2} )}^{k}} {\prod }_{i = 1}^{k} {\vert {β}_{i} \vert}^{- ( r + 1 )} exp ( - \frac{τ}{{β}_{i}^{2}} ) \qquad Equation 6$$

Here, βₖ is a vector of coefficients of length k, and r, τ>0. The hyperparameter τ represents a scale parameter that determines the dispersion of the prior around 0, while r is similar to the shape parameter in the Inverse Gamma distribution and determines the tail behavior of the density. τ and r are configurable parameters and may be varied depending on the embodiment. For example, in some embodiments, r may be set to 1 and τ may be set to 3, although other positive values are also possible. Details regarding methods for selecting appropriate values for r and τ are further discussed in Nikooienejad 2016, the full contents of which are incorporated herein.

The prior for model K p(k) may be calculated according to:

$$p ( k ) = \frac{B ( a + k , b + p - k )}{B ( a , b )} \qquad Equation 7$$

Where B(a,b) denotes the beta function and a and b are prior parameters that describe an underlying beta distribution on the marginal probability that a selected feature is associated with a non-zero regression coefficient. In some embodiments, based on the assumption that optimal predictive models are sparse, server 160 may set a=min(k*, [log (p)]), and b=p−a. For large n, this implies that we expect, on average, a feature vectors to be included in the model. Here,

$${k}^{*} = arg {max}_{k} ( \begin{matrix}p \\ k\end{matrix} ) < {2}^{n} .$$

This choice of k* for the prior hyperparameter reflects the belief that the number of models that can be constructed from available covariates should be smaller than the number of possible binary responses. Similarly, by restricting a to be less than log (p), comparatively small prior probabilities are assigned to models that contain more than log (p) covariates. Finally, server 160 may impose a deterministic constraint on model size and define P(k)=0 if k<n/2.

In general, any search algorithm for determining which models out of all possible models to explore (i.e., to calculate a posterior probability for) may be used. For instance, in some embodiments, a purely random search algorithm that selects a predetermined set of models for which to compute posterior probabilities may be used. In other embodiments, a simple birth-death process may be used to explore the model space. Such a scheme may first calculate a posterior probability for an initial model that accepts a randomly-determined set of types of digital features. At each iteration of this scheme, a number i between 1 and p is randomly selected. A candidate model is constructed by flipping the inclusion state of the i′th digital feature—that is, if the current model includes the i′th digital feature, the candidate model would not include that feature, and if the current model does include the i′th digital feature, then the candidate model would include that feature. The scheme then determines whether to move from the current model to the candidate model (i.e., whether to accept the candidate model) may be calculated according to a probability r, where r is determined by:

$$r = \frac{{m}_{k , cand} ( {y}_{n} ) p ( {k}^{cand} )}{{m}_{k , cand} ( {y}_{n} ) p ( {k}^{curr} )} \qquad Equation 8$$

Such a scheme explores the possible model space by evaluating models one at a time and determining whether to move from a current model to a candidate model at each iteration according to Equation 8. After exploring a pre-determined number N of candidate models (and calculating N different posterior probabilities), the scheme may terminate.

Returning to FIG. 4, at step 408, the server 160 may select a narrowed subset of the plurality of types of digital features based on the posterior probabilities computed for each model at step 406. This may be done by computing a score for each respective type of digital feature calculated at step 404 that is indicative of how much said respective type of digital feature contributed to the accuracy of a model for detecting events. This score may be computed based at least in part on the posterior probabilities computed for each model at step 406. For example, the score for each respective digital feature may comprise the sum of the posterior probabilities for each model that includes said respective digital feature. Such a score may also be referred to herein as a “posterior inclusion probability” or “PIP”.

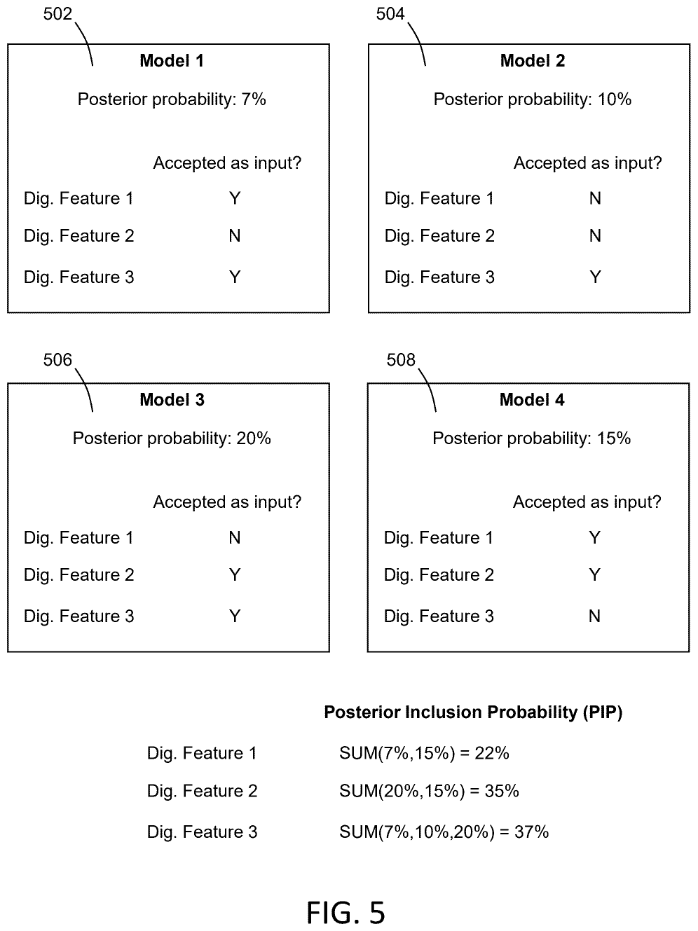

FIG. 5 presents one example for how such a PIP is computed at step 408. Consider an illustrative example where, upon the conclusion of step 406, server 160 has calculated posterior probabilities for four models. Each model accepts as input a different subset of three different digital features, denoted digital features 1, 2, and 3 in this example. Model 1 (502) has a calculated posterior probability of 7% and accepts as input digital features 1 and 3 only. Model 2 (504) has a calculated posterior probability of 10% and accepts as input digital feature 3 only. Model 3 (506) has a calculated posterior probability of 20% and accepts as input digital features 2 and 3 only. Model 4 (508) has a calculated posterior probability of 15% and accepts as input digital features 1 and 2 only. The PIP for digital feature 1 is the average of the posterior probabilities of the models that accept digital feature 1 as input. Under this example, only models 1 and 4 accept digital feature 1 as input, and so the PIP for digital feature 1 is the sum of 7% and 15% (the posterior probabilities of models 1 and 4, respectively), which is 22%. Similarly, the PIP for digital feature 2 is the sum of the posterior probabilities of the models that accept digital feature 2 as input. Under this example, only models 3 and 4 accept digital feature 2 as input, and so the probability score for digital feature 2 is the sum of 20% and 15% (the posterior probabilities of models 3 and 4, respectively), which is 35%. When the PIP for digital feature 3 is similarly calculated, the score in this example is 37%.

Once the PIPs for digital features have been selected, the narrowed subset of digital features may be selected based on such PIPs. For example, the digital features may be ranked according to their PIPs and the top M ranked digital features may be selected, where M is a configurable parameter. In some embodiments, digital features with PIPs above a certain configurable threshold may be selected. In order to reduce computational resources required to train and/or use a machine-learning model, the narrowed subset of digital features should be less than the number of digital features calculated at step 404 of FIG. 4.

In some embodiments, the server 160 may use the published R package BVSNLP (Bayesian Variable Selection in High Dimensional Settings using Nonlocal Priors), published online on Jun. 29, 2020 by Amir Nikooienejad et al, to calculate the posterior probabilities for each explored model. The BVSNLP algorithm is described in greater detail in Nikooienejad et al., “Bayesian Variable Selection for Survival Data Using Inverse Moment Priors,” Ann Appl Stat. 2020 June; 14 (2): 809-828, the full contents of which are incorporated herein. The BVSNLP algorithm was originally designed for high dimensional settings where the number of digital features, p, is much larger than the number of observations, n, that is p>>n. This makes the BVSNLP algorithm efficient in p but not in n. In some embodiments, when the BVSNLP package is used to calculate posterior probabilities according to the process described herein, the number of observations n may greatly exceed the number of digital features being evaluated. In order to improve the computational efficiency of the BVSNLP algorithm in such scenarios, the full training dataset may be randomly divided into batches (e.g., 6,000 batches) with an equal number of observations (e.g., 1,000 observations) in each batch. In each batch, the ratio of observations with a ground truth label indicating an event vs. not an event may be kept the same as the original dataset in order to prevent any potential bias. Each batch may then be processed separately by the BVSNLP method. After the BVSNLP method is done for each batch, the digital features may be ranked according to their PIP and the top X variables by PIP extracted for each batch (where X is a configurable parameter). At the end and to merge the output from all batches (e.g., all 6,000 batches), the pooled list of all selected features is grouped by variable name and the PIPs of multiple instances of a feature are averaged. Finally, the top Y variables with the highest PIPs are selected, where Y is a configurable parameter.

Referring again to FIG. 4, at step 410, a machine-learning model may be trained using only the narrowed subset of types of digital features that were selected at step 408. In this way, the computational resources and time required to train the machine-learning model may be reduced. The computational resources required to use the trained machine-learning model may also be reduced. In some embodiments, a machine-learning model that accepts as input only the narrowed subset of digital features selected using process 400 may be implemented on a mobile device, such as a smartphone, tablet, smartwatch, or portable computer.

The terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

Various aspects are described in this disclosure, which include, but are not limited to, the following aspects:

1. A computerized method for detecting whether a subject is experiencing an event using one or more processors, the method comprising: receiving time-series data derived from data recorded by an actigraphy device worn on a wrist of the subject; computing a set of birth and death coordinates for each topological feature of a plurality of topological features in the received time-series data; calculating a digital feature based on the computed set of birth and death coordinates for each topological feature in the plurality of topological features; and determining whether the time-series data indicates the subject is experiencing the event based on the calculated digital feature.

2. The method of aspect 1, wherein each topological feature in the plurality of topological features is associated with a contiguous segment of time during which the time-series data is below a filtration threshold.

3. The method of aspect 2, wherein computing the set of birth and death coordinates comprises increasing the filtration threshold to identify the set of birth and death coordinates.

4. The method of aspect 3, wherein the birth coordinate for each topological feature corresponds to a first level of the filtration threshold at which said topological feature first appears as the filtration threshold is increased.

5. The method of any of aspects 2-4, wherein the death coordinate for each topological feature specifies a second level for the filtration threshold at which said topological feature first disappears as the filtration threshold is increased.

6. The method of any of aspects 2-5, wherein a total number of topological features at a specified level for the filtration threshold corresponds to a Betti number for the time-series data at said specified level for the filtration threshold.

7. The method of any of aspects 1-6, wherein calculating the digital feature comprises calculating a lifespan persistence (L) for each topological feature in the plurality of topological features by subtracting the birth coordinate for said topological feature from the death coordinate for said topological feature.

8. The method of aspect 7, wherein the digital feature comprises at least one of a mean, a standard deviation, a skewness, a kurtosis, and an entropy of the calculated lifespan persistence (L) for each topological feature.

9. The method of any of aspects 1-6, wherein calculating the digital feature comprises calculating a midlife persistence (M) for each topological feature by calculating a mean average of the set of birth and death coordinates for said topological feature.

10. The method of aspect 9, wherein the digital feature comprises at least one of a mean, a standard deviation, a skewness, a kurtosis, and an entropy of the calculated midlife persistence (M) for each topological feature.

11. The method of any of aspects 1-10, wherein the received time-series data is derived from at least one of accelerometer data and gyroscope data.

12. The method of aspect 11, wherein the received time-series data is derived from recorded accelerometer data having an ax, ay, and az component.

13. The method of aspect 11, wherein the received time-series data is derived from recorded gyroscope data having a gx, gy, and gz component.

14. The method of any of aspects 1-13, wherein determining whether the time-series data indicates the subject is experiencing the event comprises providing the calculated digital feature to a trained machine-learning model and obtaining a prediction from said trained model regarding whether the calculated digital feature indicates the subject is experiencing the event.

15. The method of any of aspects 1-14, wherein the actigraphy device is worn on a dominant wrist of the subject.

16. The method of any of aspects 1-15, wherein the method is performed entirely on a handheld mobile device.

17. The method of any of aspects 1-16, wherein the event comprises an eating event.

18. The method of any of aspects 1-16, wherein the event comprises a scratching event.

19. A system for detecting whether a subject is experiencing an event, the system comprising: memory storing computer-executable instructions; and one or more processors configured to execute the instructions to perform the method of any of aspects 1-16.

20. The system of aspect 19, further comprising the actigraphy device.

21. Non-transitory computer-readable media storing instructions that, when executed by one or more processors, are operable to cause the one or more processors to perform the method of any of aspects 1-18.

22. A computerized method for training a machine-learning model to detect whether a subject is experiencing an event using one or more processors, the method comprising: receiving, training data comprising: a plurality of sets of time-series data derived from data recorded by an actigraphy device worn on a wrist of a subject, and a ground truth label associated with each respective set of time-series data indicating whether the respective set of data is derived from data recorded by the actigraphy device while the subject is experiencing the event; calculating a plurality of types of digital features for each set of time-series data in the plurality of sets; for each model of a plurality of models, calculating a posterior probability that said model is an optimal model for detecting whether the subject is experiencing the event given the received training data, wherein each model of the plurality of models accepts a different subset of the plurality of types of digital features as an input; selecting a narrowed subset of the plurality of types of digital features based on the calculated probabilities; and training a machine-learning model using the narrowed subset of types of digital features to detect whether the subject is experiencing the event.

23. The method of aspect 22, wherein the plurality of models is less than a number of possible subsets of the plurality of types of digital features.

24. The method of any of aspects 22-23, wherein selecting the narrowed subset based on the calculated probabilities comprises: for each type of digital feature in the plurality of types of digital features, computing a posterior inclusion probability by summing the calculated probabilities for each model of the plurality of models that uses said type of digital feature as an input; and selecting the narrowed subset based on the computed posterior inclusion probability.

25. The method of aspect 24, wherein selecting the narrowed subset based on the calculated probabilities comprises: ranking each type of digital feature according to the computed posterior inclusion probability; and selecting a predetermined number of digital features having the highest computed posterior inclusion probability.

26. The method of any of aspects 22-25, wherein the time-series data is derived from at least one of accelerometer data and gyroscope data.

27. The method of aspect 26, wherein the received time-series data is derived from recorded accelerometer data having an ax, ay, and az component.

28. The method of aspect 26, wherein the received time-series data is derived from recorded gyroscope data having a gx, gy, and gz component.

29. The method of any of aspects 22-28, wherein the actigraphy device is worn on a dominant wrist of the subject.

30. The method of any of claims 22-29, wherein the event comprises an eating event.

31. The method of any of claims 22-29, wherein the event comprises a scratching event.

32. A system for training a machine-learning model to detect whether a subject is experiencing an event, the system comprising: memory storing computer-executable instructions; and one or more processors configured to execute the instructions to perform the method of any of aspects 22-29.

33. The system of aspect 30, further comprising the actigraphy device.

34. Non-transitory computer-readable media storing instructions that, when executed by one or more processors, are operable to cause the one or more processors to perform the method of any of aspects 22-29.

1-34. (canceled)

35. An actigraphy device configured to worn on a wrist of a subject and for detecting whether the subject is experiencing an eating event, the actigraphy device comprising:
an accelerometer configured to measure raw accelerometer data indicative of the subject's movement along multiple axes;
a gyroscope configured to measure raw gyroscope data indicative of the subject's movement along multiple axes;
memory storing computer-executable instructions; and
a processor at the actigraphy device configured to execute the instructions to:
derive time-series data from the raw accelerometer data and the raw gyroscope data;
compute a set of birth and death coordinates for each topological feature of a plurality of topological features in the derived time-series data;
calculate a digital feature based on the computed set of birth and death coordinates for each topological feature in the plurality of topological features; and
determine whether the time-series data indicates the subject is experiencing the eating event based on the calculated digital feature.

36. The device of claim 35, further comprising a communication device configured to establish a wireless communication link with a mobile device.

37. The device of claim 35, wherein each topological feature in the plurality of topological features is associated with a contiguous segment of time during which the time-series data is below a filtration threshold.

38. The device of claim 37, wherein computing the set of birth and death coordinates comprises increasing the filtration threshold to identify the set of birth and death coordinates.

39. The device of claim 38, wherein the birth coordinate for each topological feature corresponds to a first level of the filtration threshold at which said topological feature first appears as the filtration threshold is increased.

40. The device of claim 37, wherein the death coordinate for each topological feature specifies a second level for the filtration threshold at which said topological feature first disappears as the filtration threshold is increased.

41. The device of claim 37, wherein a total number of topological features at a specified level for the filtration threshold corresponds to a Betti number for the time-series data at said specified level for the filtration threshold.

42. The device of claim 35, wherein calculating the digital feature comprises calculating a lifespan persistence (L) for each topological feature in the plurality of topological features by subtracting the birth coordinate for said topological feature from the death coordinate for said topological feature.

43. The device of claim 42, wherein the digital feature comprises at least one of a mean, a standard deviation, a skewness, a kurtosis, and an entropy of the calculated lifespan persistence (L) for each topological feature.

44. The device of claim 35, wherein calculating the digital feature comprises calculating a midlife persistence (M) for each topological feature by calculating a mean average of the set of birth and death coordinates for said topological feature.

45. The device of claim 44, wherein the digital feature comprises at least one of a mean, a standard deviation, a skewness, a kurtosis, and an entropy of the calculated midlife persistence (M) for each topological feature.

46. The device of claim 35, wherein determining whether the time-series data indicates the subject is experiencing the eating event comprises providing the calculated digital feature to a trained machine-learning model and obtaining a prediction from said trained model regarding whether the calculated digital feature indicates the subject is experiencing the eating event.

47. The device of claim 35, wherein the actigraphy device is configured to be worn on a dominant wrist of the subject.

48. The device of claim 35, wherein the actigraphy device is further configured to detect scratching events.
